SEQUENCE LISTING (11 sequences)

SEQ ID NO: 1 - DNA, 592 bp, Arabidopsis thaliana
ataaactaagggcgtctcatactctcatatatatttgcatctaatcttgtaagcaaacgttatcacttgtctacacaacattctttcatttacaataataatatgggtgtaacattagaaggacaaagaaaggaatcaatttgggttttgatgagaagacaaagggctcgaagggcacttgtgaagaagatcatgatccgaccaaggaagagtgtagaagcttctagaagaccttgtcgcgcaatacacagacgagtgaagacgctaaaagagcttgttcccaacaccaaaacatcagaaggtttagatggactctttagacaaacggcagattatatcttggctttggaaatgaaagtgaaagttatgcagacaatggttcaggttttgaccgaaactaactgtgtttaaaagccttcatatattttttgtatatcttgttggattttacgttctttttagtttttatttgttcgtgtttattttttattatctcgtgtgattgtcttgtgttgcttatatagaaaaggaatttggtttatcttgctgctgtagactatgcagaaaattaaatatcaaaaatatatatgta

SEQ ID NO: 2 - DNA, 309 bp, Arabidopsis thaliana
  CDS (1)..(309)
atgggtgtaacattagaaggacaaagaaaggaatcaatttgggttttgatgagaagacaaagggctcgaagggcacttgtgaagaagatcatgatccgaccaaggaagagtgtagaagcttctagaagaccttgtcgcgcaatacacagacgagtgaagacgctaaaagagcttgttcccaacaccaaaacatcagaaggtttagatggactctttagacaaacggcagattatatcttggctttggaaatgaaagtgaaagttatgcagacaatggttcaggttttgaccgaaactaactgtgtttaa

SEQ ID NO: 3 - protein, 102 aa, Arabidopsis thaliana
MGVTLEGQRKESIWVLMRRQRARRALVKKIMIRPRKSVEASRRPCRAIHRRVKTLKELVPNTKTSEGLDGLFRQTADYILALEMKVKVMQTMVQVLTETNCV

SEQ ID NO: 4 - RNA, 309 nt, Arabidopsis thaliana
auggguguaacauuagaaggacaaagaaaggaaucaauuuggguuuugaugagaagacaaagggcucgaagggcacuugugaagaagaucaugauccgaccaaggaagaguguagaagcuucuagaagaccuugucgcgcaauacacagacgagugaagacgcuaaaagagcuuguucccaacaccaaaacaucagaagguuuagauggacucuuuagacaaacggcagauuauaucuuggcuuuggaaaugaaagugaaaguuaugcagacaaugguucagguuuugaccgaaacuaacuguguuuaa

SEQ ID NO: 5 - DNA, 21 bp, Arabidopsis thaliana
gttatgcagacaatggttcag

SEQ ID NO: 6 - DNA, 22 bp, Arabidopsis thaliana
agcaacacaagacaatcacacg

SEQ ID NO: 7 - DNA, 154 bp, Arabidopsis thaliana
gttatgcagacaatggttcaggttttgaccgaaactaactgtgtttaaaagccttcatatattttttgtatatcttgttggattttacgttctttttagtttttatttgttcgtgtttattttttattatctcgtgtgattgtcttgtgttgct

SEQ ID NO: 8 - DNA, 21 bp, Arabidopsis thaliana
atgggtgtaacattagaagga

SEQ ID NO: 9 - DNA, 27 bp, Arabidopsis thaliana
aacacagttagtttcggtcaaaacctg

SEQ ID NO: 10 - DNA, 306 bp, Arabidopsis thaliana
atgggtgtaacattagaaggacaaagaaaggaatcaatttgggttttgatgagaagacaaagggctcgaagggcacttgtgaagaagatcatgatccgaccaaggaagagtgtagaagcttctagaagaccttgtcgcgcaatacacagacgagtgaagacgctaaaagagcttgttcccaacaccaaaacatcagaaggtttagatggactctttagacaaacggcagattatatcttggctttggaaatgaaagtgaaagttatgcagacaatggttcaggttttgaccgaaactaactgtgtt

SEQ ID NO: 11 - protein, 84 aa, Oryza sativa
MPPPASNHGAACKNSGSNKERRRMRRCVEVRRKMEALRRLVPGGGGGEDSGEELLFRAADYIARLQVQVKVMQLMVDVLEQTKD

(19) United States
(12) Patent Application Publication
(10) Pub. No.: US 2009/0271894 A1
(43) Pub. Date: Oct. 29, 2009

(54) COMPOSITIONS AND METHODS FOR MODULATING BIOMASS IN ENERGY CROPS

(75) Inventors: Philip N. Benfey, Chapel Hill, NC (US); Hironaka T. Tsukagoshi, Durham, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Duke University

(21) Appl. No.: 12/429,013

(22) Filed: Apr. 23, 2009

Related U.S. Application Data
(60) Provisional application No. 61/047,334, filed on Apr. 23, 2008.

(51) Int. Cl. C12N 15/82; A01H 5/00; C12P 7/06; C12Q 1/68

(52) U.S. Cl. 800/287; 800/290; 800/320; 435/161; 435/6

(57) ABSTRACT

Methods for modulating plant biomass are provided. In some embodiments, the methods include the step of modulating in a plant cell the expression of an UPBEAT1 gene product. Also provided are improved energy crop plants, and seeds and parts thereof, which contain a heterologous nucleic acid that encodes an UPBEAT1 gene product and/orthat encodes an inhibitor of an UPBEAT1 gene product.

### CROSS REFERENCE TO RELATED APPLICATIONS

The presently disclosed subject matter claims the benefit of U.S. Provisional Patent Application Ser. No. 61/047,334, filed Apr. 23, 2008, the disclosure of which is incorporated herein by reference in its entirety.

### GOVERNMENT INTEREST

This presently disclosed subject matter was made with U.S. Government support under Grant No. 0618304 awarded by United States National Science Foundation. Thus, the U.S. Government has certain rights in the presently disclosed subject matter.

### TECHNICAL FIELD

The presently disclosed subject matter relates to methods for modifying biomass in a plant. Also provided are methods for increasing biomass in a plant, improved plants with modified biomass, methods for producing ethanol from the improved plants, and methods for identifying mutagenized seeds.

### BACKGROUND

In plants, stem cell centers are located in the meristem, which can be found at the tip of the root and shoot. These stem cells divide to give progeny that undergo a series of rapid divisions, after which the cells begin to expand. The process of cell expansion dramatically increases the volume of the cell, often at least a hundred fold. It is during this expansion that large amounts of cellulose are deposited around each expanding cell.

Identification of genes that will increase biomass and cellulose in combination with promoters that will drive these genes in the appropriate locations is a step toward engineering better energy crops. The improvement of yield in maize and other cereals has been dramatic with intensive breeding over the last 50 years. The environmental necessity of moving rapidly to alternative fuel sources reduces the effectiveness of conventional breeding, as performed for maize, to increase yields of energy crops.

Perennial grasses like switchgrass and Miscanthus are promising cellulosic ethanol feed stocks since they can grow on marginal land, require relatively little water and fertilizer, produce abundant biomass, prevent soil erosion and can potentially sequester large amounts of carbon. Nonetheless, cellulosic ethanol is not yet cost-competitive with conventional gasoline. A component in making energy crops competitive with current energy sources is to increase their agronomic productivity.

Switchgrass is a promising energy crop because it is native to North America and has the potential to thrive on dormant and farm land. It is also a desirable energy crop because the plants will never enter the food supply.

Brachypodium distachyon is emerging as a leading model for energy crop improvement. It is a member of the same family of plants as switchgrass, but it is better suited to molecular and genetic studies because it is a diploid annual, it has a short (4 month) life cycle, and it can be transformed with relative ease. It also has a small genome (160 Mb), which is being sequenced by the United States Department of Energy (DOE) Joint Genome Initiative. With respect to transgenic analysis, it is worth noting that Brachypodium transformation is now as efficient as rice transformation and is currently being applied to functional genomic studies (Garvin et al., 2008).

What is needed is an improved energy crop plant with increased cellulosic biomass per acre. The results of the investigation described herein, show that by modulating how and when cell expansion occurs, it is possible to increase the size of plants, thus increasing their biomass and cellulose content.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for modifying biomass in a plant. In some embodiments, the methods comprise modulating in one or more cells in a plant the expression of an UPBEAT1 gene. In some embodiments, the UPBEAT1 gene comprises at least 25 contiguous nucleotides of SEQ ID NO: 2. In some embodiments, the modulating comprises down regulating the expression of the UPBEAT1 gene. In some embodiments, the down regulating comprises inducing RNA interference targeted to a transcription product of the UPBEAT1 gene. In some embodiments,

The presently disclosed subject matter also provides methods for increasing biomass in a transgenic plant. In some embodiments, the methods comprise expressing in the transgenic plant a heterologous nucleic acid encoding an inhibitor of an UPBEAT1 gene product. In some embodiments, the transgenic plant comprises a construct comprising a promoter that is active in the transgenic plant operably linked to the heterologous nucleic acid encoding the inhibitor of the UPBEAT1 gene product. In some embodiments, the promoter is selected from the group consisting of (a) a promoter that is specific to cell division; (b) a promoter that is specific to cell elongation; and (c) a tissue-specific promoter. In some embodiments, the UPBEAT1 gene product is encoded by a nucleotide sequence comprising SEQ ID NO: 2.

The presently disclosed subject matter also provides improved plants comprising a transgene construct comprising a promoter operably linked to a nucleic acid encoding an inhibitor of translation of an UPBEAT1 gene product. In some embodiments, the improved plant is a transgenic plant selected from the group consisting of switchgrass, Miscanthus, and sorghum. In some embodiments, the inhibitor of translation of the UPBEAT1 gene product is selected from the group consisting of an miRNA targeted to a transcription product of the UPBEAT1 gene, an siRNA targeted to a transcription product of the UPBEAT1 gene, and an antisense UPBEAT1 sequence. In some embodiments, the UPBEAT1 gene product comprises SEQ ID NO: 3 or is encoded by a nucleic acid comprising SEQ ID NO: 2.

The presently disclosed subject matter also provides methods for producing ethanol. In some embodiments, the methods comprise (a) obtaining biomass from an improved plant comprising a transgene construct comprising a promoter operably linked to a nucleic acid encoding an inhibitor of an UPBEAT1 gene product; (b) treating the biomass to render carbohydrates in the biomass fermentable; and (c) fermenting the carbohydrates to produce ethanol. In some embodiments, the improved plant is a transgenic switchgrass. In some embodiments, the inhibitor of the UPBEAT1 gene product is selected from the group consisting of an miRNA targeted to a transcription product of the UPBEAT1 gene, an siRNA targeted to a transcription product of the UPBEAT1 gene, and an antisense UPBEAT1 sequence. In some embodiments, the UPBEAT1 gene product comprises SEQ ID NO: 3 or is encoded by a nucleic acid comprising SEQ ID NO: 2.

The presently disclosed subject matter also provides methods for identifying a mutagenized seed comprising a mutation in a UPBEAT1 (UPB1) gene. In some embodiments, the methods comprise mutagenizing one or more seeds using a mutagen and identifying a mutagenized seed using SEQ ID NO: 1 or SEQ ID NO: 2 as a reference sequence. In some embodiments, the identifying comprises (a) mutagenizing a plurality of seeds with a mutagen; (b) pooling DNA samples from the mutagenized plurality of seeds and/or from plants generated therefrom; (c) amplifying a sequence of interest from each of the pooled DNA samples, the sequence of interest comprising a nucleotide sequence of or transcribed from the UPB1 locus in the mutagenized seeds; and (d) identifying within at least at least one amplified sequence of interest one or more mutations in a nucleic acid molecule in the mutagenized seeds, whereby a mutagenized seed comprising a mutation in a UPB1 gene is identified. In some embodiments, the mutation in the UPB1 gene downregulates UPB1 biological activity in a plant comprising the mutation relative to a plant that comprises a wild type UPB1 gene. In some embodiments, the mutagen is a random chemical mutagen, optionally ethane methyl sulfonate (EMS).

The presently disclosed subject matter also provides methods for producing ethanol comprising (a) obtaining biomass from a plant comprising a mutation in an UPB1 gene, wherein expression of the UPB1 gene is reduced in the plant relative to wild type plant of the same species that does not comprise the mutation in the UPB1 gene; (b) treating the biomass to render carbohydrates in the biomass fermentable; and (c) fermenting the carbohydrates to produce ethanol.

Thus, it is an object of the presently disclosed subject matter to provide methods and compositions for modifying biomass in a plant.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE SEQUENCE LISTING

SEQ ID NO: 1 is nucleotide sequence of a cDNA that corresponds to an UPBEAT1 gene from Arabidopsis.

SEQ ID NO: 2 is a sequence of an open reading frame of an UPBEAT1 gene from Arabidopsis. It corresponds to nucleotides 103-411 of SEQ ID NO: 1.

SEQ ID NO: 3 is an amino acid sequence encoded by SEQ ID Nos: 1 and 2.

SEQ ID NO: 4 is a sequence of an RNA encoded by SEQ ID NO: 2.

SEQ ID NO: 5 is a nucleotide sequence of a forward primer that can be used in conjunction with a reverse primer having the nucleotide sequence of SEQ ID NO: 6 in qRT-PCR to produce an amplified product having SEQ ID NO: 7.

SEQ ID NO: 8 is a nucleotide sequence of a forward primer that can be used in conjunction with a reverse primer having the nucleotide sequence of SEQ ID NO: 9 in qRT-PCR to produce an amplified product having SEQ ID NO: 10.

SEQ ID NO: 11 is an amino acid sequence of a rice ortholog of UPB1.

### DETAILED DESCRIPTION

The presently disclosed subject matter relates generally to methods for improving energy crops. In some embodiments the presently disclosed subject matter pertains to modulating the behavior of genes in energy crops that control cell expansion with a goal of increasing biomass and cellulose content.

Specific promoter and gene combinations are described which regulate cell expansion and thus increase biomass. In the case of UPBEAT1 (UPB1) as one non-limiting example, which negatively influences plant growth, RNAi and artificial microRNAs were fused to different promoters to reduce UPBEAT1 gene expression and increase plant growth.

To elaborate, modulation of UPB1 expression alters plant size, biomass, and cellulose in energy crops. Modulation, such as but not limited to down regulation, can be achieved such as but not limited to by RNA interference (RNAi), for example by microRNAs and/or siRNAs targeted to UPB1 gene products. Methods and compositions are described regarding the modulation of UPB1 expression in Arabidopsis, Brachypodium, and switchgrass, in some embodiments.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a marker” refers to one or more markers. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

As used herein, the term “or” is used in the “inclusive” sense of “and/or” and not the “exclusive” sense of “either/or”, unless specifically indicated otherwise.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “allele” refers to any of one or more alternative forms of a gene, all of which relate to at least one trait or characteristic. In a diploid cell, two alleles of a given gene occupy corresponding loci on a pair of homologous chromosomes, although one of ordinary skill in the art understands that the alleles in any particular individual do not necessarily represent all of the alleles that are present in the species.

The term “AtGenExpress” refers to a multinational effort designed to uncover the transcriptome of the multicellular model organism A. thaliana. It can be accessed through the website of The Arabidopsis Information Resource (TAIR) on the World Wide Web.

As used herein, the term “backcross”, and grammatical variants thereof, refers to a process in which a breeder crosses a progeny individual back to one of its parents, for example, a first generation hybrid F1 with one of the parental genotypes of the F1 hybrid. In some embodiments, a backcross is performed repeatedly, with a progeny individual of one backcross being itself backcrossed to the same parental genotype.

As used herein, the term “basic Helix-Loop-Helix” (bHLH) refers to a protein structural motif found in transcription factors having two α-helices connected by a loop.

The term “chromosome” is used herein in its art-recognized meaning of the self-replicating genetic structure in the cellular nucleus containing the cellular DNA and bearing in its nucleotide sequence the linear array of genes.

As used herein, the term “Columbia-0” (Col-0) refers to a widely used wild type variety of Arabidopsis thaliana. Col-0 contains no visible genetic markers and is commonly used in mapping and mutation studies and as a basis for measurement comparisons, for example for root length and meristem size.

As used herein, the terms “cultivar” and “variety” refer to a group of similar plants that by structural or genetic features and/or performance can be distinguished from other varieties within the same species.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the term “gene” refers to a hereditary unit including a sequence of DNA that occupies a specific location on a chromosome and that contains the genetic instruction for a particular characteristics or trait in an organism.

As used herein, the term “gene fusion” refers to a hybrid gene formed from two previously separate genes. For example, by creating a fusion gene of a protein of interest and green fluorescent protein, the protein of interest can be observed in cells or tissue using fluorescence microscopy.

As used herein, the term “Gene Ontology” (GO), refers to a bioinformatics initiative to unify the representation of gene and gene product attributes across all species

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell and/or, if from the same source, is modified from its original form. Thus, a heterologous gene in a host cell includes a gene that is endogenous to the particular host cell but has been modified, for example by mutagenesis and/or by isolation from native transcriptional regulatory sequences. The terms also include non-naturally occurring multiple copies of a naturally occurring nucleotide sequence. Thus, the terms refer in some embodiments to a DNA segment that is foreign or heterologous to the cell, or is homologous to the cell but in a position within the host cell nucleic acid wherein the element is not ordinarily found.

As used herein, the term “heterozygous” refers to a genetic condition existing when different alleles reside at corresponding loci on homologous chromosomes.

As used herein, the term “homozygous” refers to a genetic condition existing when identical alleles reside at corresponding loci on homologous chromosomes.

As used herein, the term “hybrid” in the context of nucleic acids refers to a double-stranded nucleic acid molecule, or duplex, formed by hydrogen bonding between complementary nucleotide bases. The terms “hybridize” or “anneal” refer to the process by which single strands of nucleic acid sequences form double-helical segments through hydrogen bonding between complementary bases.

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous individual or line.

As used herein, the terms “introgression”, “introgressed”, and “introgressing” refer to both a natural and artificial process whereby genomic regions of one species, variety, or cultivar are moved into the genome of another species, variety, or cultivar, by crossing those species. The process can optionally be completed by backcrossing to the recurrent parent.

As used herein, the term “locus” refers to a position that a given gene or a regulatory sequence occupies on a chromosome of a given species.

The term “M2”, in seed mutagenesis, refers to progeny derived from the self-fertilization of M1 individuals. The M1 generation are the individuals actually treated with the mutagen. Only dominant mutations are detected in the M1. The M2 generation is the first generation following mutagenesis in which homozygous recessive mutations can be detected, and for this reason, it is the generation most frequently used in screening for mutants.

The terms “microRNA” and “miRNA” are used interchangeably and in some embodiments can refer to synthetic or single-stranded RNA molecules, of 17-24 nucleotides in length, which regulate gene expression. By way of example and not limitation, a primary transcript (pri-miRNA) is processed to give rise to a short-stem-loop pre-miRNA, which can be further processed to produce an miRNA, which is a single-stranded RNA molecule of 17-24 nucleotides. By way of example and not limitation, the miRNA is partially complementary to a subsequence of one or more mRNA transcripts, and can downregulate expression of genes encoded by the transcripts with which there is an interaction.

The term “modulate” refers to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” or “suppress”, but the use of the word “modulate” is not limited to this definition.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Rossolini et al. (1994) Mol Cell Probes 8:91-98). The terms “nucleic acid” or “nucleic acid sequence” can also be used interchangeably with gene, open reading frame (ORF), cDNA, and mRNA encoded by a gene.

As used herein, the phrase “nucleotide sequence homology” refers to the presence of homology between two polynucleotides. Polynucleotides have “homologous” sequences if the sequence of nucleotides in the two sequences is the same when aligned for maximum correspondence. Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence homology” for polynucleotides, such as 50, 60, 70, 80, 90, 95, 98, 99 or 100 percent sequence homology can be determined by comparing two optimally aligned sequences over a comparison window, wherein the portion of the polynucleotide sequence in the comparison window may include additions or deletions (i.e., gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms, or by visual inspection. Readily available sequence comparison and multiple sequence alignment algorithms are, respectively, the Basic Local Alignment Search Tool (BLAST; Altschul et al., 1990; Altschul et al., 1997) and ClustaIX (Chenna et al., 2003) programs, both available on the internet. Other suitable programs include, but are not limited to, GAP, BestFit, PlotSimilarity, and FASTA, which are part of the Accelrys GCG Package available from Accelrys, Inc. of San Diego, Calif., United States of America.

As used herein, the term “offspring” plant refers to any plant resulting as progeny from a vegetative or sexual reproduction from one or more parent plants or descendants thereof. For instance an offspring plant can be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, and the like.) are specimens produced from selfings of F1s, F2s and the like. An F1 can thus be (and in some embodiments is) a hybrid resulting from a cross between two true breeding parents (true-breeding is homozygous for a trait), while an F2 can be (and in some embodiments is) an offspring resulting from self-pollination of the F1 hybrids.

The terms “operatively linked” and “operably linked”, as used herein, refer to a nucleic acid molecule in which a promoter region is connected to a nucleotide sequence in such a way that the transcription of that nucleotide sequence is controlled and regulated by the promoter region. Similarly, a nucleotide sequence is said to be under the “transcriptional control” of a promoter to which it is operably linked. Techniques for operatively linking a promoter region to a nucleotide sequence are known in the art. As used herein, the term “phenotype” refers to a detectable characteristic of a cell or organism, which characteristics are at least partially a manifestation of gene expression.

As used herein, the phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “population” refers to a genetically heterogeneous collection of plants sharing a common genetic derivation.

As used herein, the term “primer” refers to an oligonucleotide which is capable of annealing to a nucleic acid target allowing a DNA polymerase to attach, thereby serving as a point of initiation of DNA synthesis when placed under conditions in which synthesis of a primer extension product is induced (e.g., in the presence of nucleotides and an agent for polymerization such as DNA polymerase and at a suitable temperature and pH).

The primer (in some embodiments an extension primer and in some embodiments an amplification primer) is in some embodiments single stranded for maximum efficiency in extension and/or amplification. In some embodiments, the primer is an oligodeoxyribonucleotide.

A primer is typically sufficiently long to prime the synthesis of extension and/or amplification products in the presence of the agent for polymerization. The minimum lengths of the primers can depend on many factors, including, but not limited to temperature and composition (A/T vs. G/C content) of the primer.

In the context of an amplification primer, these are typically provided as a pair of bi-directional primers consisting of one forward and one reverse primer as commonly used in the art of DNA amplification such as in PCR amplification.

As such, it will be understood that the term “primer”, as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing.

Primers can be prepared by any suitable method. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066.

Primers can be labeled, if desired, by incorporating detectable moieties by for instance spectroscopic, fluorescence, photochemical, biochemical, immunochemical, or chemical moieties.

Template-dependent extension of an oligonucleotide primer is catalyzed by a polymerizing agent in the presence of adequate amounts of the four deoxyribonucleotide triphosphates (dATP, dGTP, dCTP and dTTP; i.e., dNTPs) or analogues, in a reaction medium that comprises appropriate salts, metal cations, and a pH buffering system. Suitable polymerizing agents are enzymes known to catalyze primer- and template-dependent DNA synthesis. Known DNA polymerases include, for example, E. coli DNA polymerase I or its Klenow fragment, T4 DNA polymerase, and Taq DNA polymerase, as well as various modified versions thereof. The reaction conditions for catalyzing DNA synthesis with these DNA polymerases are known in the art. The products of the synthesis are duplex molecules consisting of the template strands and the primer extension strands, which include the target sequence. These products, in turn, can serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount can vary, and is determined by the function which the product polynucleotide is to serve.

The PCR method is well described in handbooks and known to the skilled person. After amplification by PCR, the target polynucleotides can be detected by hybridization with a probe polynucleotide which forms a stable hybrid with that of the target sequence under stringent to moderately stringent hybridization and wash conditions. If it is expected that the probes will be essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell, 2001. Generally, lower salt concentration and higher temperature increase the stringency of hybridization conditions.

Continuing, the term “probe” refers to a single-stranded oligonucleotide sequence that will form a hydrogen-bonded duplex with a complementary sequence in a target nucleic acid sequence analyte or its cDNA derivative.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al., 1994; Yu et al., 1999), and the MUC1 promoter (Kurihara et al., 2000) as discussed above, as well as other tissue-specific and cell-type specific promoters known to those of skill in the art.

As used herein, the term “q-value” refers to the minimum false positive rate at which an individual hypothesis test is statistically significant.

As used herein, the term “recombination” refers to an exchange (a “crossover”) of DNA fragments between two DNA molecules or chromatids of paired chromosomes over a region of similar or identical nucleotide sequences. A “recombination event” is herein understood to refer to a meiotic crossover.

As used herein, the term “regenerate”, and grammatical variants thereof, refers to the production of a plant from tissue culture.

The term “ribozyme”, also known as “RNA enzyme” or “catalytic RNA” refers to ribonucleotides or RNA molecules that can act as enzymes that catalyze covalent changes in the structure of RNA molecules and that can cleave the target RNA molecule.

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, “RNA interference” (RNAi) refers to a process of sequence-specific post-transcriptional gene silencing. See generally Fire et al., 1998; and U.S. Pat. No. 6,506,559. RNA interference (RNAi) is a natural process by which living cells can control which genes are expressed or suppressed by inhibiting an RNA molecule and stopping or at least substantially reducing the expression of the protein encoded by this RNA molecule. If the target protein has a function in the cell, RNAi approaches can result in loss of that function. As such, RNAi technology is an attractive therapeutic tool to modulate the expression of genes. RNAi can be mediated by several natural and synthetic constructs, including double stranded RNA (dsRNA), or smaller dsRNA known as small interfering RNAs (siRNA), short hairpin RNA (shRNA), microRNA (miRNA), or synthetic hammerhead ribozymes. These can be referred to as examples of RNAi molecules.

The process of RNA interference (RNAi) mediated post-transcriptional gene silencing is thought to be an evolutionarily conserved cellular defense mechanism that has evolved to prevent the expression of foreign genes (Fire, Trends Genet 15:358-363, 1999). RNAi might have evolved to protect cells and organisms against the production of double stranded RNA (dsRNA) molecules resulting from infection by certain viruses (particularly the double stranded RNA viruses or those viruses for which the life cycle includes a double stranded RNA intermediate) or the random integration of transposon elements into the host genome via a mechanism that specifically degrades single stranded RNA or viral genomic RNA homologous to the double stranded RNA species.

The presence of long dsRNAs in cells stimulates the activity of the enzyme Dicer, a ribonuclease III. Dicer catalyzes the degradation of dsRNA into short stretches of dsRNA referred to as small interfering RNAs (siRNA) (Bernstein et al., Nature 409:363-366, 2001). The small interfering RNAs that result from Dicer-mediated degradation are typically about 21-23 nucleotides in length and contain about 19 base pair duplexes. After degradation, the siRNA is incorporated into an endonuclease complex referred to as an RNA-induced silencing complex (RISC). The RISC is capable of mediating cleavage of single stranded RNA present within the cell that is complementary to the antisense strand of the siRNA duplex. According to Elbashir et al., cleavage of the target RNA occurs near the middle of the region of the single stranded RNA that is complementary to the antisense strand of the siRNA duplex (Elbashir et al., Genes Dev 15:188-200, 2001b).

RNAi has been described in several cell type and organisms. Fire et al., 1998 described RNAi in C. elegans. Wianny & Zernicka-Goetz, Nature Cell Biol 2:70-75, 1999 disclose RNAi mediated by dsRNA in mouse embryos. Hammond et al., Nature 404:293-296, 2000 were able to induce RNAi in Drosophila cells by transfecting dsRNA into these cells. Elbashir et al. Nature 411:494-498, 2001a demonstrated the presence of RNAi in cultured mammalian cells including human embryonic kidney and HeLa cells by the introduction of duplexes of synthetic 21 nucleotide RNAs.

Other studies have indicated that a 5′-phosphate on the target-complementary strand of a siRNA duplex facilitate siRNA activity and that ATP is utilized to maintain the 5′-phosphate moiety on the siRNA (Nykanen et al., Cell 107:309-321, 2001). Other modifications that might be tolerated when introduced into an siRNA molecule include modifications of the sugar-phosphate backbone or the substitution of the nucleoside with at least one of a nitrogen or sulfur heteroatom (PCT International Publication Nos. WO 00/44914 and WO 01/68836) and certain nucleotide modifications that might inhibit the activation of double stranded RNA-dependent protein kinase (PKR), specifically 2′-amino or 2′-O-methyl nucleotides, and nucleotides containing a 2′-O or 4′-C methylene bridge (Canadian Patent Application No. 2,359,180).

Other references disclosing the use of dsRNA and RNAi include PCT International Publication Nos. WO 01/75164 (in vitro RNAi system using cells from Drosophila and the use of specific siRNA molecules for certain functional genomic and certain therapeutic applications); WO 01/36646 (methods for inhibiting the expression of particular genes in mammalian cells using dsRNA molecules); WO 99/32619 (methods for introducing dsRNA molecules into cells for use in inhibiting gene expression); WO 01/92513 (methods for mediating gene suppression by using factors that enhance RNAi); WO 02/44321 (synthetic siRNA constructs); WO 00/63364 and WO 01/04313 (methods and compositions for inhibiting the function of polynucleotide sequences); and WO 02/055692 and WO 02/055693 (methods for inhibiting gene expression using RNAi).

The terms “short hairpin RNA” and “shRNA” are used interchangeably and refer to any nucleic acid molecule capable of generating siRNA.

As used herein, the terms “silence”, “ablate”, “inhibit”, “suppress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. In some embodiments, inhibition results in an expression level of a gene product that is below that level observed in the absence of the modulator.

The terms “small interfering RNA”, “short interfering RNA” and “siRNA” are used interchangeably and refer to any nucleic acid molecule capable of mediating RNA interference (RNAi) or gene silencing. See e.g., Bass, Nature 411:428-429, 2001; Elbashir et al., Nature 411:494-498, 2001a; and PCT International Publication Nos. WO 00/44895, WO 01/36646, WO 99/32619, WO 00/01846, WO 01/29058, WO 99/07409, and WO 00/44914.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances. Longer sequences hybridize specifically at higher temperatures. An extensive guide to the hybridization of nucleic acids is found in Tijssen, 1993. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For selective or specific hybridization, a positive signal is in some embodiments at least two times background, and in some embodiments 10 times background hybridization. Exemplary stringent hybridization conditions include: 50% formamide, 5×SSC, and 1% SDS, incubating at 42° C.; or 5×SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al. 1989; Ausubel et al. 1999).

As used herein, the term “T-DNA” refers to transferred DNA of the Ti plasmid of Agrobacterium tumefaciens. The bacterium inserts a DNA fragment of the Ti plasmid into the genome of the plant host, in this case A. thaliana. The Ti plasmid normally induces crown gall tumors. The T-DNA interrupts the gene into which it has been transferred. The Arabidopsis T-DNA collection is a group of Col-0 plant lines with T-DNA inserts in different genes used in genetic studies.

The term “transcription factor” generally refers to a protein that modulates gene expression by interaction with the transcriptional regulatory element and cellular components for transcription, including RNA polymerase, Transcription Associated Factors (TAFs), chromatin-remodeling proteins, and any other relevant protein that impacts gene transcription.

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

### II. Methods of the Presently Disclosed Subject Matter

In accordance with the presently disclosed subject matter, genes have been identified that are associated with the regulation of cell expansion. Datasets of the presently disclosed subject matter include high-resolution gene expression profiles obtained from fine sections along the developmental axis of the root of Arabidopsis (see Brady et al., 2007 for a description of how the microarray expression profiles were generated). Several candidate genes have been identified that were up-regulated at the boundary of the meristem and elongation zones.

Additional gene expression datasets can be generated in B. distachyon. Phylogenetic approaches can be used to identify a Brachypodium ortholog(s) of UPB1 based on sequence homology. The orthologs are validated by demonstrating the expected expression pattern along the root longitudinal axis using qRT-PCR. As an initial check of function, putative orthologs are tested to see if they complement an Arabidopsis upb1 mutant. Function in Brachypodium can be assessed by analyzing transgenics overexpressing or underexpressing the candidate orthologs. In the case of underexpression, RNAi or artificial microRNAs are used to down-regulate expression. Cell division and cell size in roots, and overall biomass and cellulose content in whole plants, are assessed.

In Brachypodium, genes regulating cell expansion can be identified by analyzing gene expression along the longitudinal axis of roots. The analysis is done by tag profiling with ILLUMINA® sequencing (Illumina Inc. San Diego, Calif., United States of America) although alternate technologies are available. mRNA can be isolated from longitudinal sections of Brachypodium roots. Transcripts that show differential expression at the boundary of the meristematic and elongation zones are identified by tag profiling. Adequate resolution of sampled slices is assessed by demonstrating that dominant expression patterns from cluster analysis are contained in more than one lateral zone on average. The expression patterns of candidate genes are confirmed by use of quantitative reverse transcription-polymerase chain reaction (qRT-PCR) and their functions are assessed by transgenic analyses as described herein. For genes identified to regulate cell expansion, optimization of biomass and cellulose traits conferred by these genes is achieved through fine-tuning the expression of said genes with specific promoters.

Promoters that are specific to the zone of cell division, the zone of cell elongation as well as tissue-specific promoters can be identified and tested for their response to a range of environmental stimuli using ROOTARRAY™ technology (GrassRoots Biotechnology, Durham, N.C., United States of America). Promoters that confer appropriate expression patterns and that are unresponsive to environmental stimuli can be fused to the genes controlling cell elongation and introduced into plants. Their effects on biomass and cellulose content can be assayed.

For UPB1, where growth is negatively regulated by the gene of interest, RNAi and artificial microRNAs can be fused to different promoters to reduce gene expression and increase growth. The effects of different candidate genes paired with a range of promoters can be tested in Brachypodium and then in energy crops or other plants. Evaluation of the promoter/gene combinations on plant growth and development is performed with a real-time image analysis platform that captures the dynamics of cell division and cell elongation. Plants with altered growth characteristics are analyzed for wet and dry weight and cellulose content. The extent of improvement in biomass and cellulose production is evaluated. Some combinations of gene and promoter can result in a significant increase in biomass and cellulose.

The characterization of one of these candidate genes, UPBEAT1 (UPB1), is now provided by way of further illustration and not limitation. UPB1 is a previously uncharacterized transcription factor. Down regulation of UPB1 results in a larger plant while up regulation results in a shorter plant. A more detailed analysis of upb1 mutants in Arabidopsis has shown that cell number was increased in the meristematic zone and both cell number and cell length were increased in the elongation zone. Similar changes occurred in the shoots and leaves. Thus, UPB1 acted like a rheostat in controlling the transition from rapid cell division to cell expansion, and consequently, in controlling the size of the plant. No obvious fitness problems were observed in the larger plants.

The expression of genes that regulate cell expansion can be modulated using specific promoters. In some embodiments,. promoters specific to the zone of cell division are fused to genes controlling cell elongation and introduced into plants to modulate biomass.

In some embodiments, promoters specific to the zone of cell elongation are fused to genes controlling cell elongation and introduced into plants to modulate biomass.

In some embodiments, tissue-specific promoters are fused to genes controlling cell elongation and introduced into plants to modulate biomass. The cellulose content of the plant can thus be assayed by the use of any of these promoters described above. The promoters are identified and tested for their response to an environmental stimulus using ROOTARRAY™ technology. Promoters are selected to be unresponsive to environmental stimuli and for their gene expression patterns.

Specifically, in the case of UPB1, which negatively regulates plant growth, nucleic acids encoding an RNAi and/or an artificial microRNA targeted to a UPB1 gene product can be operatively linked to a promoter to reduce gene expression and increase plant growth. This includes fusing a nucleic acid encoding a UPB1 inhibitor (e.g., a nucleic acid encoding an miRNA, an siRNA, or an antisense UPB1 sequence) to a promoter that is specific to the zone of cell division, a promoter that is specific to the zone of cell elongation, and/or a tissue-specific promoter.

### III. Production of UPB1 Modulated Plants by Transgenic Methods

According to another aspect of the presently disclosed subject matter, a nucleic acid (in some embodiments a DNA) sequence comprising SEQ ID NO: 2, or biomass-modulating parts thereof, can be used forthe production of plants with increased biomass of the presently disclosed subject matter. In this aspect, the presently disclosed subject matter provides for the use of UPB1, or biomass-modulating parts thereof, for producing a plant with increased biomass, which use involves the introduction of a nucleic acid sequence comprising the UPB1 into a suitable recipient plant.

The nucleic acid sequence that comprises UPB1 can be transferred to a suitable recipient plant by any method available. For instance, the nucleic acid sequence can be transferred by crossing an UPB1 mutant donor plant with a wild type recipient plant (i.e., by introgression), by transformation, by protoplast fusion, by a doubled haploid technique, by embryo rescue, or by any other nucleic acid transfer system, optionally followed by selection of offspring plants comprising the presently disclosed biomass increasing allele. For transgenic methods of transfer, a nucleic acid sequence comprising a biomass increasing allele can be isolated from the donor plant using methods known in the art, and the thus isolated nucleic acid sequence can be transferred to the recipient plant by transgenic methods, for instance by means of a vector, in a gamete, or in any other suitable transfer element, such as a ballistic particle coated with the nucleic acid sequence.

Plant transformation generally involves the construction of an expression vector that will function in plant cells. In the presently disclosed subject matter, such a vector comprises a nucleic acid sequence that comprises an UPB1 mutant allele associated with increased biomass, which vector can comprise a biomass increasing gene that is under control of or operatively linked to a regulatory element, such as a promoter. The expression vector can contain one or more such operably linked gene/regulatory element combinations, provided that at least one of the genes contained in the combinations encodes UPB1. The vector(s) can be in the form of a plasmid, and can be used, alone or in combination with other plasmids, to provide transgenic plants that have increased biomass, using transformation methods known in the art, such as the Agrobacterium transformation system.

Expression vectors can include at least one marker gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the marker gene). Many commonly used selectable marker genes for plant transformation are known in the art, and include, for example, genes that code for enzymes that metabolically detoxify a selective chemical agent that can be an antibiotic or a herbicide, or genes that encode an altered target which is insensitive to the inhibitor. Several positive selection methods are known in the art, such as mannose selection. Alternatively, marker-less transformation can be used to obtain plants without the aforementioned marker genes, the techniques for which are also known in the art.

One method for introducing an expression vector into a plant is based on the natural transformation system of Agrobacterium (see e.g., Horsch et al., 1985). A. tumefaciens and A. rhizogenes are plant pathogenic soil bacteria that genetically transform plant cells. The Ti and Ri plasmids of A. tumefaciens and A. rhizogenes, respectively, carry genes responsible for genetic transformation of the plant (see e.g., Kado, 1991). Methods of introducing expression vectors into plant tissue include the direct infection or co-cultivation of plant cells with Agrobacterium tumefaciens (Horsch et al., 1985). Descriptions of Agrobacterium vectors systems and methods for Agrobacterium-mediated gene transfer are provided for example by Gruber & Crosby, 1993 and U.S. Pat. No. 5,591,616. General descriptions of plant expression vectors and reporter genes and transformation protocols and descriptions of Agrobacterium vector systems and methods for Agrobacterium-mediated gene transfer can be found in Gruber & Crosby, 1993. General methods of culturing plant tissues are provided for example by Miki et al., 1993 and by Phillips et al., 1988. A reference handbook for molecular cloning techniques and suitable expression vectors is Sambrook & Russell, 2001.

Another method for introducing an expression vector into a plant is based on microprojectile-mediated transformation wherein DNA is carried on the surface of microprojectiles. The expression vector is introduced into plant tissues with a biolistic device that accelerates the microprojectiles to speeds of 300 to 600 m/s which is sufficient to penetrate plant cell walls and membranes (see e.g., Sanford et al., 1987; Klein et al., 1988; Sanford, 1988; Sanford, 1990; Klein et al., 1992; Sanford et al., 1993). Another method for introducing DNA to plants is via the sonication of target cells (see Zhang et al., 1991). Alternatively, liposome or spheroplast fusion can be used to introduce expression vectors into plants (see e.g., Deshayes et al., 1985 and Christou et al., 1987). Direct uptake of DNA into protoplasts using CaCl₂ precipitation, polyvinyl alcohol, or poly-L-ornithine has also been reported (see e.g., Hain et al. 1985 and Draper et al., 1982). Electroporation of protoplasts and whole cells and tissues has also been described (D'Halluin et al., 1992 and Laursen et al., 1994).

Other well known techniques such as the use of BACs, wherein parts of a plant genome are introduced into bacterial artificial chromosomes (BACs), i.e., vectors used to clone DNA fragments (100- to 300-kb insert size; average, 150 kb) in Escherichia coli cells, based on a naturally occurring F-factor plasmid found in the bacterium E. coli. (Zhao & Stodolsky, 2004) can be employed for example in combination with the BIBAC system (Hamilton, 1997) to produce transgenic plants.

Following transformation of plant target tissues, expression of the above described selectable marker genes allows for preferential selection of transformed cells, tissues and/or plants, using standard regeneration and selection methods.

### IV. Production of Increased Biomass Plants by Non-Transgenic Methods

In some embodiments for producing an increased biomass plant, protoplast fusion can be used for the transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, which can even be obtained with plant species that cannot be interbred in nature, is tissue cultured into a hybrid plant exhibiting the desirable combination of traits. More specifically, a first protoplast can be obtained from a plant that exhibits increased biomass. A second protoplast can be obtained from a second plant variety, preferably a plant line that comprises commercially valuable characteristics, such as, but not limited to disease resistance, insect resistance, valuable nutritional characteristics, and the like. The protoplasts are then fused using traditional protoplast fusion procedures, which are known in the art.

Alternatively, embryo rescue can be employed in the transfer of a nucleic acid comprising one or more biomass increasing loci as described herein from a donor plant to a recipient plant. Embryo rescue can be used as a procedure to isolate embryos from crosses wherein plants fail to produce viable seed. In this process, the fertilized ovary or immature seed of a plant is tissue cultured to create new plants (Pierik, 1999).

The presently disclosed subject matter also relates to methods for producing an increased biomass plant comprising performing a method for detecting the presence of an allele associated with increased biomass in a donor plant according to the presently disclosed subject matter as described above, and transferring a nucleic acid sequence comprising at least one allele thus detected, or a biomass increasing part thereof, from the donor plant to a recipient plant. The transfer of the nucleic acid sequence can be performed by any of the methods previously described herein.

An exemplary embodiment of such a method comprises the transfer by introgression of the nucleic acid sequence from an increased biomass donor plant into a recipient plant by crossing the plants. This transfer can thus suitably be accomplished by using traditional breeding techniques. Biomass increasing loci are introgressed in some embodiments into commercial plant varieties using marker-assisted selection (MAS) or marker-assisted breeding (MAB). MAS and MAB involve the use of one or more of the molecular markers for the identification and selection of those offspring plants that contain one or more of the genes that encode for the desired trait. MAB can also be used to develop near-isogenic lines (NIL) harboring the gene of interest, allowing a more detailed study of each gene effect. MAB is also an effective method for development of backcross inbred line populations (see e.g., Nesbitt & Tanksley, 2001; van Berloo et al., 2001). Plants developed according to these embodiments can advantageously derive a majority of their traits from the recipient plant, and derive increased biomass from the donor plant.

As discussed hereinabove, traditional breeding techniques can be used to introgress a nucleic acid sequence encoding increased biomass into a recipient plant. In some embodiments, a donor plant that exhibits increased biomass and comprising a nucleic acid sequence encoding UPB1 is crossed with a recipient plant that in some embodiments exhibits commercially desirable characteristics, such as, but not limited to, disease resistance, insect resistance, valuable nutritional characteristics, and the like. The resulting plant population (representing the F1 hybrids) is then self-pollinated and allowed to set seeds (F2 seeds). The F2 plants grown from the F2 seeds are then screened for increased biomass. The population can be screened in a number of different ways.

First, the population can be screened using a traditional disease screen. Such disease screens are known in the art. In some embodiments, a quantitative bioassay is used. Second, marker-assisted breeding can be performed using one or more of the herein-described molecular markers to identify those progeny that comprise a nucleic acid sequence encoding for increased biomass. Other methods, referred to hereinabove by methods for detecting the presence of an allele associated with increased biomass, can be used. Also, marker-assisted breeding can be used to confirm the results obtained from the quantitative bioassays, and therefore, several methods can also be used in combination.

Inbred increased biomass plant lines can be developed using the techniques of recurrent selection and backcrossing, selfing, and/or dihaploids, or any other technique used to make parental lines. In a method of recurrent selection and backcrossing, increased biomass can be introgressed into a target recipient plant (the recurrent parent) by crossing the recurrent parent with a first donor plant, which differs from the recurrent parent and is referred to herein as the “non-recurrent parent”. The recurrent parent is a plant without increased biomass or has a low level of increased biomass and possesses commercially desirable characteristics, such as, but not limited to disease resistance, insect resistance, valuable nutritional characteristics, and the like. In some embodiments, the non-recurrent parent exhibits increased biomass and comprises a nucleic acid sequence that encodes increased biomass. The non-recurrent parent can be any plant variety or inbred line that is cross-fertile with the recurrent parent.

The progeny resulting from a cross between the recurrent parent and non-recurrent parent are backcrossed to the recurrent parent. The resulting plant population is then screened forthe desired characteristics, which screening can occur in a number of different ways. For instance, the population can be screened using phenotypic pathology screens or quantitative bioassays as known in the art. Alternatively, instead of using bioassays, marker-assisted breeding (MAB) can be performed using one or more of the hereinbefore described molecular markers, hybridization probes, or polynucleotides to identify those progeny that comprise a nucleic acid sequence encoding increased biomass. Also, MAB can be used to confirm the results obtained from the quantitative bioassays. In some embodiments, the markers defined herein are suitable to select proper offspring plants by genotypic screening.

Following screening, the F1 hybrid plants that exhibit an increased biomass phenotype or, in some embodiments, genotype and thus comprise the requisite nucleic acid sequence encoding increased biomass, are then selected and backcrossed to the recurrent parent for a number of generations in order to allow for the plant to become increasingly inbred. This process can be performed for two, three, four, five, six, seven, eight, or more generations. In principle, the progeny resulting from the process of crossing the recurrent parent with the increased biomass non-recurrent parent are heterozygous for one or more genes that encode increased biomass.

In general, a method of introducing a desired trait into a hybrid plant variety can comprise:
(a) crossing an inbred parent plant with another plant that comprises one or more desired traits, to produce F1 progeny plants, wherein the desired trait is increased biomass;
(b) selecting the F1 progeny plants that have the desired trait to produce selected F1 progeny plants, in some embodiments using molecular markers as defined herein;
(c) backcrossing the selected progeny plants with the inbred parent plant to produce backcross progeny plants;
(d) selecting for backcross progeny plants that have the desired trait and morphological and physiological characteristics of the inbred parent plant, wherein the selection comprises the isolation of genomic DNA, and testing the DNA for the presence of at least one molecular marker for increased biomass, in some embodiments as described herein;
(e) repeating steps (c) and (d) two or more times in succession to produce selected third or higher backcross progeny plants;
(f) optionally selfing selected backcross progeny in order to identify homozygous plants; and
(g) crossing at least one of the backcross progeny or selfed plants with another inbred parent plant to generate a hybrid plant variety with the desired trait and all of the morphological and physiological characteristics of hybrid plant variety when grown in the same environmental conditions.

As indicated, the last backcross generation can be selfed in order to provide for homozygous pure breeding (inbred) progeny for increased biomass. Thus, the result of recurrent selection, backcrossing, and selfing is the production of lines that are genetically homogenous for the genes associated with increased biomass, and in some embodiments as well as for other genes associated with traits of commercial interest.

### V. Increased Biomass Plants and Seeds

An improved plant, which may include an improved energy or forage crop plant, comprising a transgene construct comprising a promoter operably linked to a nucleic acid encoding an inhibitor of an UPBEAT1 gene product is an aspect of the presently disclosed subject matter.

In some embodiments, the promoters employed are plant promoters, lessening the likelihood of adverse effects from genetic engineering.

Thus, a goal of plant breeding is to combine in a single variety or hybrid various desirable traits. For commercial plants, these traits can include increased bioimass, resistance to diseases and insects, tolerance to heat and drought, reducing the time to crop maturity, greater yield, and better agronomic quality. Uniformity of plant characteristics such as germination and stand establishment, growth rate, maturity, and plant height can also be of importance.

Commercial plants are bred through techniques that take advantage of the plant's method of pollination. A plant is self-pollinated if pollen from one flower is transferred to the same or another flower of the same plant. A plant is sib pollinated when individuals within the same family or line are used for pollination. A plant is cross-pollinated if the pollen comes from a flower on a different plant from a different family or line.

Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of heterogeneous plants that differ genetically and will not be uniform.

The development of a hybrid plant variety in a plant breeding program can, in some embodiments, involve three steps: (1) the selection of plants from various germplasm pools for initial breeding crosses; (2) the selfing of the selected plants from the breeding crosses for several generations to produce a series of inbred lines, which, individually breed true and are highly uniform; and (3) crossing a selected inbred line with an unrelated inbred line to produce the hybrid progeny (F1). After a sufficient amount of inbreeding, successive filial generations will merely serve to increase seed of the developed inbred. In some embodiments, an inbred line comprises homozygous alleles at about 95% or more of its loci.

An important consequence of the homozygosity and homogeneity of the inbred lines is that the hybrid created by crossing a defined pair of inbreds will always be the same. Once the inbreds that create a superior hybrid have been identified, a continual supply of the hybrid seed can be produced using these inbred parents and the hybrid plants can then be generated from this hybrid seed supply.

An increased biomass plant, or a part thereof, obtainable by a method of the presently disclosed subject matter is an aspect of the presently disclosed subject matter.

Another aspect of the presently disclosed subject matter relates to an increased biomass plant, or part thereof, comprising the disclosed increased biomass loci in any configuration as described in detail above wherein at least one of the disclosed increased biomass loci is not in its natural genetic background. The increased biomass plants of the presently disclosed subject matter can be heterozygous or homozygous for the increased biomass traits (in some embodiments, homozygous). Although the increased biomass loci of the presently disclosed subject matter, as well as increased biomass-conferring parts thereof, can be transferred to any plant in order to provide for an increased biomass plant, the methods and plants of the presently disclosed subject matter are in some embodiments related to energy crop plants.

The increased biomass plant lines described herein can be used in additional crossings to create modified plants. For example, a first increased biomass plant of the presently disclosed subject matter can be crossed with a second plant possessing commercially desirable traits such as, but not limited to, disease resistance, insect resistance, desirable nutritional characteristics, and the like. In some embodiments, this second plant line itself has increased biomass. In some embodiments, this line is heterozygous or homozygous for one or more of the disclosed increased biomass loci, in order for one or more of these traits to be expressed in the hybrid offspring plants.

Another aspect of the presently disclosed subject matter relates to a method of producing seeds that can be grown into increased biomass plants. In some embodiments, the method comprises providing an increased biomass plant of the presently disclosed subject matter, crossing the plant with another plant, and collecting seeds resulting from the cross, which when planted, produce plants having increased biomass.

In some embodiments, the method comprises providing an increased biomass plant of the presently disclosed subject matter, crossing the increased biomass plant with a plant, collecting seeds resulting from the cross, regenerating the seeds into plants, selecting plants for increased biomass by any of the methods described herein, self-pollinating the selected plants for a sufficient number of generations to obtain plants that are fixed for an allele associated with increased biomass in the plants, backcrossing the plants thus produced with plants having desirable phenotypic traits for a sufficient number of generations to obtain plants that have increased biomass and have other desirable phenotypic traits, and collecting the seeds produced from the plants resulting from the last backcross, which when planted, produce plants which have increased biomass.

### VI. Identification of UPB1 Mutations via TILLING

A method of identifying mutations in UPB1 that reduce UPB1 expression, and which would also avoid GMO issues, is through Targeting Induced Local Lesions IN Genomes (TILLING). TILLING refers to a method in molecular biology that allows directed identification of mutations in a specific gene. The method combines a standard technique, mutagenesis with a chemical mutagen such as ethyl methane sulfonate (EMS), with a sensitive DNA screening-technique that identifies single base mutations in a target gene. Typically, in plants, seeds are mutagenized by treatment with EMS, but other chemicals such as sodium azide and/or methylnitrosourea (MNU) can be used in species where EMS proves less effective. One approach has been to try a range of treatment severity and select the treatment that gives a desired amount (typically 30%-40%) of M2 families segregating for embryo and seedling lethality (Till et al., 2006).

DNA samples taken from individuals within a mutagenized population are pooled and screened (McCallum et al., 2000). The sequence of interest is compared against a known sequence database for similarities using informatics tools such as CODDLE (available from the website of the proWEB Project). The method identifies an approximately 1,500-bp region of the target sequence that has the highest likelihood of producing mutations that will have detrimental effects on the gene. These predictions are based on the ability of a mutation to create either a nonsense codon, an alteration of a transcript splicing site, or a nonconservative missense mutation in a highly conserved domain of the predicted protein. The selected region (or a hand designed alternative) is then PCR amplified from each of the pooled DNAs and screened enzymatically for the presence of mismatched bases that result if one member of a pool carries a mutation that the other members do not, thus allowing the identification of a particular mutant individual within a pool. The target is then reamplified from specific, putative mutants, and sequenced to identify the specific base that has been altered. TILLING can return allelic series for specific genes, with a wide variety of phenotypic effects. (See e.g. Weil 2009).

EcoTILLING is a variation of TILLING which looks at natural variation by examining a cultivar/inbred line/accession against a reference genome in a one-on-one comparison to the reference genome, usually one that has been sequenced. SNPs are detected in the same manner that induced mutations are in EMS TILLING. TILLING and EcoTILLING reveal point mutations and naturally occurring SNPs that affect amino acid sequence. Plants created via TILLING are not classified as genetically modified organisms (GMOs), thus making the technique popular in food crops.

In some embodiments, the presently disclosed subject matter provides methods of identifying plants, or parts thereof, comprising UPB1 mutations through the process of TILLING wherein UPB1-1 is used as the reference gene sequence.

VII. Production of Ethanol from Biomass

In some embodiments, the presently disclosed subject matter provides methods for producing ethanol from a biomass. In some embodiments, the methods comprise (a) obtaining biomass from an improved energy crop plant comprising a transgene construct comprising a promoter operably linked to a nucleic acid encoding an inhibitor of an UPBEAT1 gene product; (b)treating the biomass to render carbohydrates in the biomass fermentable; and (c) fermenting the carbohydrates to produce ethanol.

The overall process for the production of ethanol from biomass typically involves two steps: saccharification and fermentation. First, saccharification produces fermentable sugars from the cellulose and hemicellulose in the biomass. Second, those sugars are then fermented to produce ethanol. Thorough, detailed discussion of additional methods and protocols for the production of ethanol from biomass are reviewed in Wyman, 1999; Gong et al., 1999; Sun & Cheng, 2002; Olsson & Hahn-Hagerdal, 1996, U.S. Patent Application Publication Nos. 2008/0274528 and 2007/0250961.

VII.A. Pretreatment

Raw biomass is typically pretreated to increase porosity, hydrolyze hemicellulose, remove lignin, and reduce cellulose crystallinity, all in order to improve recovery of fermentable sugars from the cellulose polymer. As a preliminary step in pretreatment, the cellulosic material can be chipped or ground. The size of the biomass particles after chipping or grinding is typically between 0.2 and 30 mm. After chipping, a number of other pretreatment options may can used to further prepare the biomass for saccharification and fermentation, including steam explosion, ammonia fiber explosion, and acid hydrolysis.

VII.A.1. Steam Explosion

Steam explosion is a common method for pretreatment of cellulosic biomass and can increase the amount of cellulose available for enzymatic hydrolysis (see U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process causes hemicellulose degradation and lignin transformation. Addition of H₂SO₄, SO², or CO² to the steam explosion reaction can improve subsequent cellulose hydrolysis, decrease production of inhibitory compounds and lead to the more complete removal of hemicellulose (Morjanoff & Gray, 1987).

VII.A.2. Ammonia Fiber Explosion (AFEX)

In AFEX pretreatment, the biomass is treated with approximately 1-2 kg ammonia per kg dry biomass for approximately 30 minutes at pressures of 1.5 to 2 MPa. (see U.S. Pat. Nos. 4,600,590; 5,037,663; Mes-Hartree et al., 1988). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun & Cheng, 2002).

VII.A.3. Acid Hydrolysis

Concentrated or dilute acids can also be used for pretreatment of biomass. H₂SO₄ and HCl have been used at high (e.g., >70%) concentrations. In addition to pretreatment, concentrated acid can also be used for hydrolysis of cellulose (see U.S. Pat. No. 5,972,118). Dilute acids can be used at either high (>160° C.) or low (<160° C.) temperatures, although high temperature is typically employed for cellulose hydrolysis (Sun & Cheng, 2002). H₂SO₄ and HCl at concentrations of 0.3 to 2% (w/w) and treatment times ranging from minutes to 2 hours or longer can be used for dilute acid pretreatment.

Other pretreatments include alkaline hydrolysis, oxidative delignification, organosolv process, or biological pretreatment (see Sun & Cheng, 2002).

VII.B. Saccharification

After pretreatment, the cellulose in the biomass can be hydrolyzed with cellulase enzymes. Cellulase catalyzes the breakdown of cellulose to release glucose which can then be fermented into ethanol.

Bacteria and fungi produce cellulases suitable for use in ethanol production (Duff & Murray, 1995). For example, Cellulomonas fimi and Thermomonospora fusca have been extensively studied for cellulase production. Among fungi, members of the Trichoderma genus, and in particular Trichoderma reesi, have been the most extensively studied. Numerous cellulases are available from commercial sources as well. Cellulases are usually actually a mixture of several different specific activities. First, endoglucanases create free chain ends of the cellulose fiber. Exoglucanases remove cellobiose units from the free chain ends and β-glucosidase hydrolyzes cellobiose to produce free glucose.

Reaction conditions for enzymatic hydrolysis are typically around pH 4.8 at a temperature between 45 and 50° C. with incubations of between 10 and 120 hours. Cellulase loading can vary from around 5 to 35 filter paper units (FPU) of activity per gram of substrate Surfactants like TWEEN® 20, 80, polyoxyethylene glycol, or TWEEN® 81 can also be used during enzyme hydrolysis to improve cellulose conversion. Additionally, combinations or mixtures of available cellulases and other enzymes can also lead to increased saccharification.

Aside from enzymatic hydrolysis, cellulose can also be hydrolyzed with weak acids or hydrochloric acid (Lee et al., 1999).

VII.C. Fermentation

Once fermentable sugars have been produced from the biomass, those sugars can be used to produce ethanol via fermentation. Fermentation processes for producing ethanol from biomass are extensively reviewed in Olsson & Hahn-Hagerdal, 1996. Briefly, for maximum efficiencies, both pentose sugars from the hemicellulose fraction of the biomass (e.g., xylose) and hexose sugars from the cellulose fraction (e.g. glucose) should be utilized. Saccharomyces cerevisiae are widely used for fermentation of hexose sugars. Pentose sugars, released from the hemicellulose portion of the biomass, can be fermented using genetically engineered bacteria, including Escherichia coli (see U.S. Pat. No. 5,000,000) or Zymomonas mobilis (Zhang et al., 1995). Fermentation with yeast strains is typically optimal around temperatures of 30 to 37° C.

VII.D. Simultaneous Saccharification and Fermentation (SSF)

Cellulase activity is inhibited by its end products, cellobiose and glucose. Consequently, as saccharification proceeds, the build up of those end products increasingly inhibits continued hydrolysis of the cellulose substrate. Thus, the fermentation of sugars as they are produced in the saccharification process leads to improved efficiencies for cellulose utilization (see e.g., U.S. Pat. No. 3,990,944). This process is known as simultaneous saccharification and fermentation (SSF), and is an alternative to the above described separate saccharification and fermentation steps. In addition to increased cellulose utilization, SSF also eliminates the need for a separate vessel and processing step. The optimal temperature for SSF is around 38° C., which is a compromise between the optimal temperatures of cellulose hydrolysis and sugar fermentation. SSF reactions can proceed up to 5 to 7 days.

VII.E. Distillation

The final step for production of ethanol is distillation. The fermentation or SSF product is distilled using conventional methods producing ethanol, for example, 95% ethanol.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

### Isolation of UPBEAT1 Gene.

Data from a gene expression map for A. thaliana that shows where and when about 22,000 of the plant's roughly 28,000 genes are activated within the growing root tissue (referred to herein as “RootMap”) were used to find transcription factors that regulate rapid cell expansion. The RootMap data comprised high-resolution gene expression profiles obtained from fine sections along the developmental axis of the Arabidopsis root (Brady et al., 2007). In some embodiments, transcription factors regulating cell expansion are expressed at the boundary between the meristem and elongation zones of the root. About 100 transcription factors were selected that showed a peak of gene expression at this boundary.

### Mutation of UPB1 Results in a Longer Root

Arabidopsis T-DNA insertion lines that were mutated in the genes encoding these transcription factors were screened. The root length phenotypes of these lines were measured in comparison to wild type (Col-0). One T-DNA mutant line had longer roots than the wild type. Quantitative reverse transcription-PCR (qRT-PCR) was used to confirm that the knockdown of expression of the gene, named UPBEAT1, was caused by the T-DNA insertion. The development of the mutant root meristem was investigated using confocal microscopy. Measurements were taken of the number of cortex cells in a cell file extending from the quiescent center (QC) to the first elongated cell (Loio et al., 2007). In this mutant, the meristem size measured in this way was increased significantly. In addition, the cell number of the elongation zone was increased. However, the radial pattern of root cell layers was not changed. The results showed that the mutated gene regulates the position of the transition zone (TZ) between the meristem and elongation zones.

For qRT-PCR, the forward primer was UPB1-RT-F (SEQ ID NO: 5) and the reverse primer was UPB1-RT-R (SEQ ID NO: 6). The amplification product was SEQ ID NO: 7.

### PCR Conditions

Total RNA was isolated from 5 dai plant root by using RNAeasy plant mini kit (Qiagen, Valencia, Calif.). For RT-PCR, the first strand cDNA was synthesized from total RNA with oligo(dT) 20 primers using SUPERSCRIPT III (Invitrogen). qRT-PCR reaction mixture was performed in 20 μl containing 200 nM of each primer.

PCR was initiated with denaturation at 95° C. for 10 min, followed by 40 cycles of denaturation at 95° C. for 15 sec, annealing and extension at 60° C. for 1 min. The comparative threshold cycle method was used to determine the relative mRNA levels. PDF1.2 was used as an internal reference.

The amplification product of qRT-PCR was not a complete open reading frame. To identify the complete open reading frame, TAIR database sequence was used. The UPB1 open reading frame was confirmed according to the EST data set. To confirm the open reading frame expression, semi-quantitative RT-PCR was performed using UPB1-trans-RT-F (SEQ ID NO: 8) and UPB1-trans-RT-R (SEQ ID NO: 9) as the primer set. PCR was initiated at 95° C. for 5 min, followed by 22, 26 or 30 cycles of denaturation at 95° C. for 15 sec, annealing at 60° C. for 15 sec, and extension at 72° C. for 30 sec. The amplification product was SEQ ID NO: 10.

### UPB1 Protein Binds DNA

The coding sequence of UPB1 is 309 base pairs, and encodes a polypeptide of 102 amino acids. The gene contains no introns. UPB1 encodes a basic Helix-Loop-Helix (bHLH) domain containing protein. The Arabidopsis genome contains 147 genes that could code for proteins with a bHLH domain. Based on the amino acid sequence of other bHLH proteins, UPB1 appears to belong to a new subfamily. It does not have any other identifiable functional domains other than the bHLH.

### UPB1 Orthologs in Rice

Orthologs of UPB1 are present in rice. Arabidopsis and Rice bHLH protein sequences were downloaded. Clustal X was used to align the sequences by whole amino acid sequences. The phylogenic tree was made using NJ tree software. The rice ortholog, Os05g0157400 (SEQ ID NO: 11), was the most similar protein to UPB1 in rice.

### Expression Pattern: UPB1 Acts in Nuclei in the Elongation Zone

The mRNA expression pattern of UPB1 was examined. According to the AtGenExpress database (The Arabidopsis Information Resource), UPB1 is expressed primarily in roots and petals. In the Rootmap data sets, UPB1 showed a peak of expression at the transition zone (TZ). To better understand these expression patterns, transcriptional and translational green fluorescent protein (GFP) fusions of UPB1 were created and introduced into Arabidopsis. The transcriptional fusion showed strong fluorescence in the lateral root cap close to the TZ but not in the tip of the root. Weak fluorescence in vascular tissue starting in the elongation zone was also detected. On the other hand, the translational fusion did not show detectable fluorescence in the lateral root cap or in the meristematic zone. In the elongation zone, GFP fluorescence was localized to the nucleus of all cell types. Moreover, fluorescence in the mature zone was not detected. The translational fusion was transformed into the ubp1-1 mutant, and it was determined that it complemented the mutant phenotypes (based on meristem size). Thus, this translational fusion had intact UPB1 activity. Taken together, these results showed that the UPB1 protein moved from the lateral root cap to the cell files in the elongation zone where it functioned in the nucleus.

To further investigate further UPB1 movement, the UPB1 coding region was fused to a triple yellow fluorescent protein (YFP) tag with the expectation that the triple YFP could prevent UPB1 movement due to its high molecular weight. In the pUPB1:UPB1-3×YFP plants, YFP fluorescence was detected only in the nucleus of the lateral root cap. This result is consistent with the hypothesis that UPB1 moves from the lateral root cap to cells of the elongation zone.

### Microarrays Show UPB1 Transcriptional Targets in the Elongation Zone

A series of whole genome microarray experiments were performed to identify potential transcriptional targets of UPB1. Roots were dissected into meristematic or elongation zones. Differentially expressed genes were detected based on their change in expression and a significant q-value threshold (1.5 fold change and 0.05 q-value). According to these criteria only 9 genes changed their expression in the meristemic zone. On the other hand, 1544 genes changed their expression in the elongation zone. This result is consistent with UPB1's primary role being in the elongation zone. Of the genes tested, 788 showed a greater than 1.5 fold higher mRNA level in the UPB1-1 mutant elongation zone compared with the Col-0 elongation zone.

Clusters of these genes were biologically classified into significantly enriched Gene Ontology (GO) categories. In clusters that showed increases in the UPB1-1 elongation zone, GO enriched categories were associated with carbohydate metabolism (p=10⁻⁴), cell wall (p=10⁻⁴) and phenylpropanoid biosynthesis (p=10⁻⁶), which was consistent with UPB1 regulating TZ position changes through cell wall modification. GO categories associated with peroxidase activity (p=10⁻⁵) and response to oxidative stress (ROS) (p=10⁻⁸) were also identified. Recently it has been reported that peroxidase and ROS levels in plant cells play an important role in controlling root growth (Dunand et al., 2007; Passardi et al., 2006). Dunand et al. reported that overexpression of a peroxidase caused longer roots while a knock down of peroxidase expression resulted in shorter root phenotypes (Passardi et al., 2006). Regarding ROS status, it is mostly superoxide (O₂⁻), which is regulated by NADPH oxidases, and which regulates plant cell growth, especially root hair formation (Foreman et al., 2003). Thus, UPB1 appears to regulate cell wall remodeling or meristem size by regulating the expression of peroxidase genes. Genes involved in cell cycle regulation were not overrepresented in the data sets, which leads to the conclusion that UPB1 is not involved in cell cycle regulation.

### ROS is Important for TZ Definition

To test the hypothesis that reactive oxygen species play a key role in controlling the TZ, Arabidopsis roots were stained with nitroblue tetrazolium (NBT) and o-dianisidine. NBT is widely used as an indicator of O₂⁻ and forms a dark blue formazan precipitate in contact with superoxide (Bielski et al., 1980). In the wild-type root tip, strong blue/violet color appeared in all cell types in the meristematic zone. In the elongation zone, only the vascular tissue was stained. This result indicated that superoxide accumulated in the root meristematic zone. In the UPB1-1 mutant, the cell specificity of the staining pattern did not change, but staining of the meristematic zone became broader than in wild-type plants. This provides further support that the meristematic zone increases in size in UPB1-1 roots.

### Modulation of UPB1 With H₂O₂ and KI

According to the microarray data, peroxidase genes were upregulated in UPB1-1 mutants. To further test this, roots were stained with o-dianisidine, which indicates peroxidase activitiy (not laccase activity) in the living root (Dunand et al., 2007). The staining for peroxidase activity was distributed along the entire root, but activity was lower in the meristematic zone when compared with the elongation zone. In the UPB1-1 mutant, staining became somewhat stronger than in wild type. This result was consistent with the microarray data.

H₂O₂ treatment prevents root growth but the molecular mechanisms are still unclear as to how H₂O₂ affects root growth. Five day old Arabidopsis plants were treated with 100 mM H₂O₂ for 6 hours. Meristem size became significantly smaller. Conversely, scavenging H₂O₂ by treating with KI resulted in a larger root meristem. Meristem size in the UPB1-1 mutant did not change significantly with either treatment. These results indicate that up-regulation of peroxidase genes in the UPB1-1 mutant altered H₂O₂ levels in the root.

Finally, H₂O₂ treatment caused an up-regulation of UPB1 mRNA, while KI treatment repressed UPB1 mRNA levels. These results indicate that H₂O₂ levels in the cell can regulate UPB1 expression creating a feedback loop in H₂O₂ signaling. Taken together these results provided new insight into a pathway in which ROS could regulate the TZ via UPB1 signal transduction and could function as a signal molecule.

### UPB1's Main Function is not Through Cytokinin and Auxin Signaling

Recently it was reported that the ratio of cytokinin to auxin was important to control the balance of cell division and differentiation in the root tip (Loio et al., 2008). Two key transcription factors, SHY2/IAA3 and AHK3/ARR1, were found to control this process. The arr1 mutant has a large meristem phenotype similar to the upb1-1 mutant. To test whether UPB1 regulates ARR1 or SHY2 expression or is involved in the same signal transduction pathway, ARR1 and SHY2 expression levels were analyzed in the upb1 mutant microarray data. The upb1 mutation did not appear to affect the expression of these genes. In addition, exogenous cytokinin application affects the TZ of upb1-1 in a similar fashion to that of wild type. These results indicate that UPB1 is not involved in the cytokinin signaling pathway to define the TZ.

### Analysis of Promoter/Coding Sequence Combinations.

The effects of candidate gene-promoter pairs are tested in Arabidopsis and then in energy crops. Promoter/gene combinations are evaluated for plant growth and development with a real-time image analysis platform that captures the dynamics of cell division and elongation. Plants with altered growth characteristics are analyzed for wet and dry weight and cellulose content. Some promoter/gene combinations result in a 30% increase in biomass.

### REFERENCES

All references listed in the instant disclosure, including but not limited to all patents, patent applications and publications thereof, scientific journal articles, and database entries (including but not limited to GENBANK® database entries and all annotations available therein) are incorporated herein by reference in their entireties to the extent not inconsistent herewith and to the extent that they supplement, explain, provide a background for, or teach methodology, techniques, and/or compositions employed herein.
Altschul et al. (1990) J. Mol. Biol. 215:403.
Altschul et al. (1997) Nucl. Acids Res. 25: 3389.
Ausubel et al. (1999) Short Protocols in Molecular Biology Wiley, New York, N.Y., United States of America.
Bass, (2001) Nature 411:428-429.
Batzer et al. (1991) Nucleic Acid Res 19:5081.
Bernstein et al. Nature 409:363-366, 2001.
Bielski et al. (1980) J Phys Chem 84:830-833.
Brady et al. (2007) Science 318:801-806.
Canadian Patent Application Publication No. 2,359,180.
Chenna et al. (2003). Nucleic Acids Res 31:3497-3500.
Christou et al. (1987) Proc Natl Acad Sci USA 84:3962-3966.
Deshayes et al. (1985) EMBO J 4:2731-2737.
D'Halluin et al. (1992) Plant Cell 4:1495-505.
Draper et al. (1982) Plant Cell Physiol 23,451-458.
Duff & Murray (1995) Bioresource Tech 55:1-33.
Dunand et al. (2007) New Phytol 174:332-341.
Elbashir et al. (2001a) Nature 411:494-498.
Elbashir et al. (2001b) Genes Dev. 15:180-200.
Fire et al., Nature 391:806-811, 1998.
Fire (1999) Trends Genet 15:358-363.
Foreman et al. (2003) Nature 422:442-446
Garvin et al. (2008) Crop Science 48(S1):S69-S84.
Gong et al. (1999) Adv Biochem Engng Biotech 65:207-241.
Greenberg et al. (1994) Mol Endocrinol 8:230-239.
Gruber & Crosby (1993) in Glick & Thompson (eds.) Methods in Plant Molecular Biology and Biotechnology, CRC Press, Baton Rouge, La., United States of America, pages 89-119.
Hain et al. (1985) Mol Gen Genet 199:161-168.
Hamilton (1997) Gene 200:107-116.
Hammond et al. (2000) Nature 404:293-296.
Horsch et al. (1985) Science 227:1229-1231.
Kado (1991) Crit Rev Plant Sci 10:1-32.
Klein et al. (1988) Biotechnology 6:559-563.
Klein et al. (1992) Bio/Technology 10:286-291.
Kurihara et al. (2000) J Clin Invest. September 2000;106(6):763-71.
Laursen et al. (1994).
Lee et al. (1999) Adv Biochem Engng Biotech 65:93-115.
Lee et al. (2000).
Loio et al. (2007). Cytokinins Determine Arabidopsis Root-Meristem Size by Controlling Cell Differentiation Curr. Biol. 17:678-682.
Loio et al. (2008). A Genetic Framework for the Control of Cell Division and Differentiation in the Root Meristem. Science 322:1380-1384.
McCallum et al. (2000) Plant Physiol 123:439-442.
Mes-Hartree et al. (1988) Appl Microbiol Biotechnol 29:462-468.
Miki et al. (1993) in Glick & Thompson (eds.) Methods in Plant Molecular Biology & Biotechnology, CRC Press, Baton Rouge, La., United States of America, pages 67-88.
Morjanoff & Gray (1987) Biotechnol Bioeng 29:733-741.
Nesbitt & Tanksley, (2001) Plant Physiol 127:575-583.
NyKanen et al. (2001). Cell 107:309-321.
Ohtsuka et al. (1985) J Biol Chem 260:2605-2608.
Olsson & Hahn-Hagerdal (1996) Enzyme Microb Technol 18:312-331.
Passardi et al. (2006). Planta 223:965-974.
Sun & Cheng (2002) Bioresource Technol 83:1-11.
PCT International Publication Nos. WO1999/07409; WO1999/32619; WO2000/01846; WO2000/44895; WO2000/44914; WO2000/63364; WO2001/04313; WO2001/29058; WO2001/36646; WO2001/68836; WO2001/75164; WO2001/92513; WO2002/055692; WO2002/055693; WO2002/44321
Phillips et al. (1988). Corn and Corn Improvement, 3^{rd} ed, Sprague et al. eds., Amer. Soc of Agronomy.
Pierik, (1999) In vitro Culture of Higher Plants, 4th edition. Martinus Nijhoff Publishers, Dordrecht, The Netherlands.
Rossolini et al. (1994) Mol Cell Probes 8:91-98.
Sambrook & Russell (2001) Molecular Cloning: A Laboratory Manual., Third Edition, Cold Spring Harbor Laboratory Press, Cold Spring Harbor, N.Y., United States of America.
Sanford et al. (1987) J Particulate Sci Technol 5:27-37.
Sanford (1988) Trends Biotechnol 6:299-302.
Sanford (1990) Physiologica Plantarum, 79:206-209.
Sanford et al. (1993) Meth Enzymol 217:483-509.
Sun & Cheng (2002) Bioresource Technol 83:1-11.
U.S. Patent Application Publication Nos. 2007/0250961; 2008/0274528.
U.S. Pat. Nos. 3,990,944; 4,461,648; 4,600,590; 5,000,000; 5,037,663; 5,591,616; 5,972,118.
Van Berloo et al. (2001) Mol Breeding 8:187-195.
Weil (2009) Plant Phys 149: 158-164.
Wianny & Zernicka-Goetz Nature Cell Biol 2:70-75 (1999).
Wyman (1999) Annu Rev Energy Environ 24:189-226.
Yu et al. (1999) Cancer Res 59:4200-4203.
Zhang et al. (1991) Biotechnology. 9:996-997.
Zhang et al. (1995) Science 267:240-243.
Zhao & Stodolsky, (2004) Bacterial Artificial Chromosomes. Methods in Molecular Biology Vol. 255. Humana Press Inc. Totowa, N.J., United States of America.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for modifying biomass in a plant, the method comprising modulating in one or more cells in a plantthe expression of an UPBEAT1 gene.

2. The method of claim 1, wherein the UPBEAT1 gene comprises at least 25 contiguous nucleotides of SEQ ID NO: 2.

3. The method of claim 1, wherein the modulating comprises down regulating the expression of the UPBEAT1 gene.

4. The method of claim 3, wherein the down regulating comprises inducing RNA interference targeted to a transcription product of the UPBEAT1 gene.

5. A method of increasing biomass in a transgenic plant, the method comprising expressing in the transgenic plant a heterologous nucleic acid encoding an inhibitor of an UPBEAT1 gene product.

6. The method of claim 5, wherein the transgenic plant comprises a construct comprising a promoter that is active in the transgenic plant operably linked to the heterologous nucleic acid encoding the inhibitor of the UPBEAT1 gene product.

7. The method of claim 5, wherein the promoter is selected from the group consisting of:
(a) a promoter that is specific to cell division;
(b) a promoter that is specific to cell elongation; and
(c) a tissue-specific promoter.

8. The method of claim 5, wherein the UPBEAT1 gene product is encoded by a nucleotide sequence comprising SEQ ID NO: 2.

9. An improved plant comprising a transgene construct comprising a promoter operably linked to a nucleic acid encoding an inhibitor of translation of an UPBEAT1 gene product.

10. The improved plant of claim 9, wherein the improved plant is a transgenic plant selected from the group consisting of switchgrass, Miscanthus, and sorghum.

11. The improved plant of claim 9, wherein the inhibitor of translation of the UPBEAT1 gene product is selected from the group consisting of an miRNA targeted to a transcription product of the UPBEAT1 gene, an siRNA targeted to a transcription product of the UPBEAT1 gene, and an antisense UPBEAT1 sequence.

12. The improved plant of claim 9, wherein the UPBEAT1 gene product comprises SEQ ID NO: 3 or is encoded by a nucleic acid comprising SEQ ID NO: 2.

13. A method for producing ethanol comprising:
(a) obtaining biomass from an improved plant comprising a transgene construct comprising a promoter operably linked to a nucleic acid encoding an inhibitor of an UPBEAT1 gene product;
(b) treating the biomass to render carbohydrates in the biomass fermentable; and
(c) fermenting the carbohydrates to produce ethanol.

14. The method of claim 13, wherein the improved plant is a transgenic switchgrass.

15. The method of claim 13, wherein the inhibitor of the UPBEAT1 gene product is is selected from the group consisting of an miRNA targeted to a transcription product of the UPBEAT1 gene, an siRNA targeted to a transcription product of the UPBEAT1 gene, and an antisense UPBEAT1 sequence.

16. The method crop plant of claim 13, wherein the UPBEAT1 gene product comprises SEQ ID NO: 3 or is encoded by a nucleic acid comprising SEQ ID NO: 2

17. A method of identifying a mutagenized seed comprising a mutation in a UPB1 gene, the method comprising mutagenizing one or more seeds using a mutagen and identifying a mutagenized seed, using SEQ ID NO: 1 or SEQ ID NO: 2 as a reference sequence.

18. The method of claim 17, wherein identifying comprises:
(a) mutagenizing a plurality of seeds with a mutagen;
(b) pooling DNA samples from the mutagenized plurality of seeds and/or from plants generated therefrom;
(c) amplifying a sequence of interest from each of the pooled DNA samples, the sequence of interest comprising a nucleotide sequence of or transcribed from the UPB1 locus in the mutagenized seeds; and
(d) identifying within at least at least one amplified sequence of interest one or more mutations in a nucleic acid molecule in the mutagenized seeds,
whereby a mutagenized seed comprising a mutation in a UPB1 gene is identified.

19. The method of claim 17, wherein the mutation in the UPB1 gene downregulates UPB1 biological activity in a plant comprising the mutation relative to a plant that comprises a wild type UPB1 gene.

20. The method of claim 17, wherein the mutagen is a random chemical mutagen, optionally ethane methyl sulfonate (EMS).

21. A method of producing ethanol, the method comprising:
(a) obtaining biomass from a plant comprising a UPB1 mutation, wherein the expression of the UPB1 gene is reduced in the plant relative to wild type plant of the same species that does not comprise a UPB1 mutation;
(b) treating the biomass to render carbohydrates in the biomass fermentable; and
(c) fermenting the carbohydrates to produce ethanol.
